(19) United States
(12) Patent Application Publication
(10) Pub. No.: US 2020/0000899 A1
(43) Pub. Date: Jan. 2, 2020

(54) PHARMACEUTICAL COMPOSITION FOR USE IN THE TREATMENT OF CANCER

(71) Applicant: AMPHERA B.V., DE 's Hertogenbosch (NL)

(72) Inventor: Joan Geertrudis Jacobus Victor AERTS, VB Steenbergen (NL)

(21) Appl. No.: 16/465,457

(22) PCT Filed: Dec. 4, 2017
(86) PCT No.: PCT/NL2017/050809

§ 371 Date: May 30, 2019

(30) Foreign Application Priority Data
Dec. 2, 2016 (NL) 2017918

(51) Int. Cl. A61K 39/00; A61K 39/395; C07K 16/28; A61P 35/00

(52) U.S. Cl. CPC A61K 39/0011; A61K 2039/505; C07K 16/2818; A61P 35/00; A61K 39/00117; A61K 39/001168; A61K 39/001162; A61K 39/001122; A61K 39/00115; A61K 39/001153; A61K 39/001186; A61K 39/001106; A61K 2039/5154; A61K 2039/545; C07K 2317/21; A61K 39/3955

(57) ABSTRACT

The present invention relates to an antigen composition comprising at least one mesothelioma cancer cell associated antigen and a pharmaceutically acceptable carrier for use in the treatment of cancer, in particular mesothelioma, wherein dendritic cells are loaded with said antigen composition and wherein said loaded dendritic cells are administered in combination with one or more checkpoint inhibitors, to patients. The present invention also relates to an antigen composition comprising at least two mesothelioma cancer cell associated antigens and a pharmaceutically acceptable carrier. The present invention further relates to an antigen composition comprising at least two mesothelioma cancer cell associated antigens and a pharmaceutically acceptable carrier, for use as a pharmaceutical, in particular for use in the treatment of mesothelioma.

### FIELD OF THE INVENTION

The present invention relates to an antigen composition for use in the treatment of cancer, wherein dendritic cells are loaded with said antigen composition and wherein said dendritic cells are administered to patients in combination with one or more checkpoint inhibitors. The present invention further relates to a composition comprising mesothelioma cancer cell associated antigens and to the pharmaceutical use thereof.

### BACKGROUND OF THE INVENTION

Malignant mesothelioma is a malignancy affecting mesothelial cells with pleura or peritoneum as the most common site of occurrence. The primary risk factor for the development of malignant mesothelioma is through inhalation of asbestos fibers. Although asbestos has been prohibited in western countries since the 1980-1990s, more cases in the future are expected due to the long latency period (20 to 50 years) of the disease.

Patients suffering from malignant mesothelioma have a very poor prognosis. Median survival durations range from nine to twelve months when no further chemotherapy is carried out. In case the patient is treated with chemotherapy (pemetrexed (Alimta)/cisplatin) the survival duration increases slightly to twelve to fifteen months.

To date, there is no standard curative therapy for malignant pleural mesothelioma (hereinafter: mesothelioma). Moreover, surgical approaches such as pleurectomy and extrapleural pneumonectomy result in high local recurrence rates and questionable survival benefit.

Because of the limited success of current treatments, novel therapeutic regimens are urgently needed. The potential to harness the potency and the specificity of the immune system underlies the growing interest in cancer immunotherapy. One approach to activate the patient's immune system uses dendritic cell based immunotherapy. Dendritic cell based immunotherapy aims to boost the immune system of cancer patients by enhancing tumor antigen recognition by activating cytotoxic T-cells and thus generating anti-tumor specific responses.

In this regard it is well known that dendritic cells are highly mobile and extremely potent antigen presenting cells located at strategic places where the body comes in contact with its environment. In these locations they pick up antigens and transport them to the secondary lymphoid organs where they instruct and control activation of natural killer cells, B and T-lymphocytes, and efficiently activate them against the antigens. This property makes them attractive candidates for use in therapeutic strategies against cancer. Furthermore, dendritic cells can be generated in large numbers ex vivo.

Previous studies in animal models and humans suffering from malignant mesothelioma demonstrated the induction of tumor-specific CD8⁺ T-cell responses accompanied by promising survival rates by means of dendritic cell based immunotherapy. Furthermore, in these studies it has also been shown that dendritic cell based immunotherapy is safe and well tolerated by patients suffering from malignant mesothelioma.

In these studies, an autologous tumor lysate was used to load dendritic cells for vaccination. However, this autologous technique had limitations when applied in a clinical setting because often the quality and/or quantity of the tumor material obtained from patients was insufficient to load onto dendritic cells. Due to this limitation, more than 80% of the patients were excluded from participating in this experimental clinical study.

An alternative source to load dendritic cells is the use of allogenic tumor lysate generated from mesothelioma cancer cell line cultures. On the basis of mouse studies it was shown that allogenic lysate prepared from allogenic tumor cell lines is able to induce antitumor responses when loaded onto dendritic cells. It was further established that when loaded onto dendritic cells, allogenic tumor lysate is as effective as autologous tumor lysate in stimulating CD8⁺ T cells antitumor response.

It has further been demonstrated that the efficacy of treatments with a limited number of antigens are often of short duration. Tumors are able to relatively rapidly down-regulate a specific antigen after which the treatment becomes ineffective. Furthermore, it has also been found that in several patients the effect of the treatment with loaded dendritic cells diminishes over time. Clearly, in these patients the tumor also found ways to downregulate specific antigens and/or to stage other counter measures against activated T-cells.

Hence, a need remains for an efficient curative or palliative treatment of cancer in general and malignant mesothelioma in particular.

### SUMMARY OF THE INVENTION

A first aspect of the present invention relates to an antigen composition comprising at least one mesothelioma cancer cell associated antigen and a pharmaceutically acceptable carrier for use in the treatment of cancer, wherein dendritic cells are loaded with said antigen composition and are administered to patients in need thereof together with one or more checkpoint inhibitors.

It has been found that the efficacy of immunotherapy is influenced considerably by the immunosuppressive environment created by the tumor. The presence of this immunosuppressive environment hampers attempts at harnessing the potency of the immune system. An increasing number of immunosuppressive factors and cells are reported in malignant diseases. One particular strategy tumors use, is to target the so called immune checkpoints, which act as the off-switch on the T cells of the immune system.

In order to overcome this defense mechanism of the tumor, the present inventors have found that it is beneficial to treat patients suffering from cancer and which have been subjected to dendritic cell immunotherapy with a checkpoint inhibitor, such as a PD1/PD-L1 inhibitor. Although clinical tests have been carried out with patients suffering from mesothelioma, the present inventors expect that patients suffering from other types of cancer will also benefit from this type of combination treatment.

A second aspect of the present invention relates to an antigen composition comprising at least two mesothelioma cancer cell associated antigens and a pharmaceutically acceptable carrier for use in the treatment of cancer, wherein dendritic cells are loaded with said antigens and are administered to a patient in need thereof, wherein said treatment effectively extends the progression free survival and/or overall survival of the patient.

It has also been found that tumor antigens play an important role in the generating the desired immune-response. However, because differential antigen expression takes place in different mesothelioma tumor cell lines found in patients, it appeared that it is not sufficient to provide dendritic cells loaded with just one antigen from a mesothelioma cell line. The present inventors have found that it is particularly efficient to load the dendritic cells to be used in the treatment of mesothelioma with at least two mesothelioma cancer cell associated antigens. This way, it is more likely that T-cells will recognize and invade the tumor cells.

A last aspect of the present invention relates to use of said antigen composition in the treatment of cancer, in particular in the treatment of mesothelioma.

### Definitions

The term “antigen” as used herein has its conventional meaning and refers to a molecule capable of inducing an immune response. Within the context of the present invention the antigen may be a protein or a fragment thereof, such as a (poly)peptide representing an epitope of said protein. It is however also possible that the antigen used is an artificial peptide or a peptidomimetic. The antigens used in the present invention are preferably proteins or parts thereof obtained or derived from a tumor-cell.

The term “epitope” as used herein has its conventional meaning and refers to the part of an antigen that is recognized by the immune system. Within the context of the present invention the antigen is a protein and the epitope is part thereof (i.e. a (poly)peptide, fragment or aggregate thereof).

The term “dendritic cells” as used herein has its conventional meaning and refers to antigen-presenting cells (also known as accessory cells) of the mammalian immune system.

The term “cancer” as used herein has its conventional meaning and refers to the broad class of disorders characterized by hyper-proliferative cell growth in vivo.

The term “mesothelioma cancer cells” as used herein has its conventional meaning and refers to cells from malignant mesothelioma.

The term “for use in the treatment of cancer” as used herein has its conventional meaning and refers to the reduction of the size of a tumor or number of cancer cells, cause a cancer to go into remission or prevent further growth in size or cell number of cancer cells.

The term “immune checkpoint” as used herein has its conventional meaning and refers molecules in the immune system that either turn up a signal (co-stimulatory molecules) or turn down a signal.

The term “checkpoint inhibitor” as used herein has its conventional meaning refers to any compound inhibiting the function of an immune checkpoint and typically include antibodies, peptides, nucleic acid molecules and small molecules.

The term “cold tumor” as used herein has its conventional meaning refers to a tumor wherein there is no or minimal presence of infiltrating cytotoxic T-cells.

The term “hot tumor” as used herein has its conventional meaning refers to a tumor wherein there is a considerable presence of cytotoxic T-cells either active or inactivated via for example the different immune checkpoints.

The term “progression free survival” (PFS) as used herein has its conventional meaning and refers to the time from treatment (or randomization) to first disease progression or death.

The term “overall survival” (OS) as used herein has its conventional meaning and refers to the patient remaining alive for a defined period of time, such as 1 year, about 1.5 years etc. from initiation of treatment or from initial diagnosis. In the studies underlying the present invention the event used for survival analysis was death from any cause.

The terms “extending survival” or “increasing the likelihood of survival” is meant increasing PFS and/or OS in a treated patient relative to an untreated patient (i.e. relative to a patient not treated with dendritic cells loaded with the antigen composition according to the invention) or relative to a control treatment protocol, such as treatment only with a chemotherapeutic agent, such as those used in the standard care of mesothelioma.

### DETAILED DESCRIPTION OF THE INVENTION

A first aspect of the present invention relates to an antigen composition comprising at least one mesothelioma cancer cell associated antigen and a pharmaceutically acceptable carrier for use in the treatment of cancer, wherein said dendritic cells are loaded with said antigen composition and are administered to patients in need thereof together with a checkpoint inhibitor.

It has been found that the efficacy of immunotherapy is influenced considerably by the immunosuppressive environment created by the tumor. The presence of this immunosuppressive environment hampers attempts at harnessing the potency of the immune system. An increasing number of immunosuppressive factors and cells are reported in malignant diseases. One particular strategy tumors use, is to target the so called immune checkpoints, which act as the off-switch on the T cells of the immune system.

The present inventors have found that this is also the case with mesothelioma. In clinical studies wherein dendritic cells were loaded with a cell lysate of mesothelioma tumor cells it has been observed that in several patients at first a good reaction (i.e. no progression or even reduction of the tumor) was observed, but that after several months the tumor started to grow again. It has further been found that the presence of anti-tumor directed T-cells are different between patients, depending on the stage of the disease, clinical condition etc.

Without wishing to be bound by any theory, the present inventors believe that under the influence of the treatment with antigen loaded dendritic cells the influx of tumor directed cytotoxic T-cells turns the so called “cold” mesothelioma tumor (i.e. non-inflamed) into a “hot” tumor (i.e. inflamed). However, in a considerable part of the patients this upregulation of the immune activity causes an immune escape by the tumor, for instance via the PD1/PD-L1 pathway or other checkpoints. This immune escape can be counteracted by applying checkpoint inhibitors, such as PD-1/PD-L1 checkpoint inhibitors. Although this mechanism has been validated for mesothelioma, it may also be used for other types of cancer.

The present invention thus relates to a method of treatment of patients suffering from cancer, in particular malignant mesothelioma, by loading (preferably autologous) dendritic cells with the antigen composition of the present invention and vaccinating said patients therewith and providing together therewith (preferably after said vaccination) one or more checkpoint inhibitors.

The checkpoint inhibitors to be used within the context of the present invention are preferably selected from inhibitors which inhibit (e.g. interact with) a checkpoint protein selected from the group of: TIM3, CTLA4, PD1, PD-L1, PD-L2, LAG3, CD137, CD40, OX40, VISTA, CD112R and BTLA, more preferably TIM3, CTLA4, PD1, PD-L1, PD-L2 and LAG3, most preferably PD1 and PD-L1. In this regard it is noted that it may also be advantageous to use a combination of different checkpoint inhibitors.

Promising results have been observed in clinical trials wherein dendritic cell immunotherapy was used in combination with PD1/PD-L1 inhibitors. Preferably said PD1/PD-L1 inhibitor is an antibody. Particularly preferred antibodies are atezolizumab, avelumab, durvalumab, nivolumab and pembrolizumab, preferably nivolumab.

The checkpoint inhibitor, in particular a PD1/PD-L1 inhibitor, is administered simultaneously or sequentially in either order, preferably the checkpoint inhibitor is administered after administration of the loaded dendritic cells, i.e. after vaccination.

In an embodiment of the present invention the antigen composition comprises at least two mesothelioma cancer cell associated antigens. Particularly with mesothelioma it has been observed that good results are achieved when the dendritic cells are loaded with different antigens from mesothelioma cancer cells. It has, however, also been observed that some of these antigens are also present in other (related) tumors. Hence, it is also possible to use them against other types of cancer than mesothelioma. However, it is of course preferred to use them against malignant mesothelioma.

It has further been found that it is advantageous to load the dendritic cells with more than one mesothelioma cancer cell associated antigen. Hence, preferably the composition for loading the dendritic cells comprises at least three, preferably at least five, more preferably at least ten mesothelioma cancer cell associated antigens. In this regard it is further noted that the antigens may be derived from the same protein, i.e. the antigens may be different epitopes from the same protein. However, it is preferred to use antigens which are (or are based) on different tumor cell associated proteins.

In order for the T-cells to able to attack all tumor cells it is important to make sure that the dendritic cells are loaded with antigens that cover ideally all tumor cells of a tumor. After all, if a specific tumor cell does not have a specific antigen an immune response will not be triggered against such a cell. If other cells are attacked, but this cell is not, it will have an advantage and will be able to grow further resulting in a further growth of the tumor. The inventors have now been able to establish the most important antigens which can be used to load dendritic cells and target substantially all tumor cells of in particular malignant mesothelioma. This information was not yet available in the art and has allowed the present inventors to formulate an antigen composition which is particularly useful for loading dendritic cells and inducing an immune response to mesothelioma tumor cells. It has further been found that this composition can also be used against other (related) types of cancer. In view of the defense mechanisms of the tumor, it is advantageous to use it in combination with a checkpoint inhibitor.

The mesothelioma cancer cell associated antigens are preferably chosen from the group of RAGE1/MOK, Mesothelin, EphA2, Survivin, WT1, MUC1. It has been established that these antigens are present in most mesothelioma cancer cells and are thus able to induce by means of dendritic cell immunotherapy a strong immune reaction against the tumor cells. Further antigens which are of importance within the context of the present invention are RAB38/NY-MEL-1, BING4, MAGE A12, HER-2/Neu, Glypican, LMP2.

Furthermore, with respect to these tumor cell associated proteins it is noted that as antigens also parts of these proteins (i.e. epitopes thereof) may be used for loading the dendritic cells. In this regard it is further noted that also polypeptides or peptidomimetics of such epitopes may be used for loading the dendritic cells. In one embodiment, the antigen composition comprises only antigens selected from the above mentioned group of antigens. This is advantageous from a regulatory perspective.

In another embodiment the mesothelioma cancer cell associated antigens are obtained from a lysate of allogenic mesothelioma tumor cells from at least two different mesothelioma tumor cell lines, preferably at least three tumor cell lines, more preferably at least four tumor cell lines, most preferably at least five tumor cell lines. The advantage of the use of such a lysate is that many mesothelioma associated antigens will be present in the lysate and that the dendritic cells are loaded with a considerable number of antigens, reducing the chances that a tumor cell will not be recognized and escapes the immune reaction. In this regard reference is made to the international patent application WO2014/102220 of the present inventors, which is herewith incorporated by reference.

The mesothelioma tumor cell lines used for preparing such a lysate are preferably chosen from Thorr 01 (deposit No. DSM ACC3191), Thorr 02 (deposit No. DSM ACC3192), Thorr 03 (deposit No. DSM ACC3193), Thorr 04 (deposit No. DSM ACC3194), Thorr 05 (deposit No. DSM ACC3195), Thorr 06 (deposit No. DSM ACC3196).

Said lysate is prepared from between 10*10⁶ and 50*10⁶ cells Hence, the lysate according to the present invention comprises an equivalent of between 10*10⁶ and 50*10⁶ tumor cells per ml.

It has further been found that the total protein content of the antigen composition is of relevance, as this is directly related to the number of tumor cells used for preparing the composition. If the amount of protein (i.e. antigen) is too the loading of dendritic cells will be poor and the induced immune response will be limited. If the protein concentration is too high, interactions between the different proteins will occur, making the antigens less available for absorption by the dendritic cells and causing stability problems. Hence, the total amount of protein in the antigen composition is preferably between 5 and 25 mg protein per ml, more preferably between 10 and 20 mg protein per ml.

In order to induce a sufficiently large immune response it is advantageous to administer a patient in need thereof with between 1*10⁶ to 1*10⁶ dendritic cells, preferably between 1*10⁶ to 50*10⁶ dendritic cells, most preferably between 10*10⁶ to 50*10⁶ dendritic cells per vaccination.

The dendritic cells used may be autologous or allogenic. However, in order to avoid any problems it is preferred to use autologous dendritic cells.

In order to reduce the size of the tumor as much as possible before immunotherapy, it is advantageous to subject the patients to prior surgery and/or chemotherapy. It is often observed that these methods reduce the tumor considerably, but that (small) parts of tumors remain in the body. In order to remove these parts, immunotherapy according to the present invention is particularly useful.

As is clear from the clinical trials that have been carried out, the present treatment effectively extends the progression free survival and/or overall survival of patient. This is for example clear when the progression free survival or overall survival of such patients is compared with a patient receiving chemotherapy only.

The antigen composition as such or the composition of dendritic cells loaded with the antigen composition according to the present invention are formulated together with a pharmaceutically acceptable carrier.

The term “carrier” as used herein has its conventional meaning and refers to a diluent, adjuvant, excipient, or vehicle with which the antigen composition or composition of dendritic cells is prepared, stored and/or administered. Such pharmaceutical carriers can be sterile liquids, such as water and oils, including those of petroleum, animal, vegetable or synthetic origin. Water is a preferred carrier when the pharmaceutical composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typically, compositions for intravenous administration are solutions in sterile isotonic aqueous buffer. Where necessary, the composition to be administered to the patients may also include a solubilizing agent and a local anesthetic such as lidocaine to ease pain at the site of the injection. Generally, the ingredients are supplied either separately or mixed together in unit dosage form or a kit of parts, for example, as a dry lyophilized powder or water free concentrate in a hermetically sealed container such as an ampoule or sachette indicating the quantity of the antigens. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

Preferably, the antigen composition and the composition of dendritic cells loaded therewith are formulated such that it is suitable for acting as a vaccine. The forms or methods for manufacturing vaccine compositions according to the present invention are not particularly limited, and a composition in a desired form can be prepared by applying a single method available in the field of the art or methods in an appropriate combination. For the manufacture of a vaccine composition, aqueous media such as distilled water for injection and physiological saline, as well as one or more kinds of pharmaceutical additives available in the field of the art can be used. For example, buffering agents, pH adjusting agents, solubilizing aids, stabilizing agents, soothing agents, antiseptics and the like can be used, and specific ingredients thereof are well known to those skilled in the art. The composition can also be prepared as a solid preparation such as a lyophilized preparation, and then prepared as an injection by adding a solubilizing agent such as distilled water for injection before use.

A second aspect of the present invention relates to an antigen composition comprising at least two mesothelioma cancer cell associated antigens and a pharmaceutically acceptable carrier for use in the treatment of cancer, preferably mesothelioma, wherein dendritic cells are loaded with said antigens and are administered to a patient in need thereof, wherein said treatment effectively extends the progression free survival and/or overall survival of the patient.

Clinical trials have shown that this approach is very beneficial to patients suffering from cancer, in particular mesothelioma. The progression free survival increased significantly. For example. it may be as much as 14.6 months or more, such as 15 months or even 24 months or more for a group of patients suffering from mesothelioma.

Also the overall survival of such patients increased significantly. For example and median overall survival for a group of patients may be 21.3 months or more, such as 22 months, 24 months or more. This is in any case much better than the expected progression free survival and/or overall survival of patients treated with chemotherapy only, such as pemetrexed/cisplatin.

Hence, the increase in overall survival may be at least about 6.3 months or more when compared to another patient treated with said chemotherapy only.

The present inventors have now for the first time identified the most relevant antigens which are present in mesothelioma tumor cells and which antigens may thus be used for loading dendritic cells and for generating an immune reaction via T-cells against the tumor cells. The composition of the present invention may thus be used for loading dendritic cells. It is preferably used in the treatment of mesothelioma, although it may also be used in the treatment of other (related) types of cancer.

Preferably, antigen composition according to the present invention comprises at least two mesothelioma cancer cell associated antigens.

More preferably, the antigen composition comprises at least three, preferably at least five, more preferably at least ten mesothelioma cancer cell associated antigens.

It is advantageous to load the dendritic cells with at least the most relevant antigens. The present inventors have been able to establish these.

Hence, in a further preferred embodiment the antigen composition comprises mesothelioma cancer cell associated antigens chosen from the group of: RAGE1/MOK, Mesothelin, EphA2, Survivin, WT1, MUC1.

Further antigens which may be used are RAGE1/MOK, Mesothelin, EphA2, Survivin, WT1, MUC1, RAB38/NY-MEL-1, BING4, MAGE A12, HER-2/Neu, Glypican, LMP2.

Furthermore, the mesothelioma cancer cell associated antigens may be obtained from a lysate of allogenic mesothelioma tumor cells from at least two different cell lines, preferably at least three cell lines, more preferably at least four cell lines, most preferably at least five cell lines. In this regard reference is made to the international patent application WO2014/102220 of the present inventors, which is herewith incorporated by reference.

Most preferably, said antigens are obtained from the mesothelioma tumor cell lines Thorr 01 (deposit No. DSM ACC3191), Thorr 02 (deposit No. DSM ACC3192), Thorr 03 (deposit No. DSM ACC3193), Thorr 04 (deposit No. DSM ACC3194), Thorr 05 (deposit No. DSM ACC3195), Thorr 06 (deposit No. DSM ACC3196).

Said lysate is preferably prepared from between 10*10⁶ and 50*10⁶ cells Hence, the lysate according to the present invention comprises an equivalent of between 10*10⁶ and 50*10⁶ tumor cells per ml.

It has further been found that the total protein content of the antigen composition is of relevance as this has a direct relationship with the number of cells used for preparing said lysate. If the amount of protein (i.e. antigen) is too low the loading of dendritic cells will be poor and the induced immune response will be limited. If the protein concentration is too high, interactions between the different proteins will occur, making the antigens less available for absorption by the dendritic cells and causing stability problems. Hence, the total amount of protein in the antigen composition is preferably between 5 and 25 mg protein per ml, more preferably between 10 and 20 mg protein per ml.

The present invention will be elucidated further by means of the following non-limiting examples.

### EXAMPLES

### Example 1: Treatment of Patients with Autologous Dendritic Cells Loaded with Antigen Composition According to the Present Invention

Patients

Patients with advanced mesothelioma were included in a phase I clinical (bridging) trial. These patients were either treatment naive, or were non-progressing after chemotherapy. Leuckapheresis was performed to obtain an enriched monocyte fraction from which immature dendritic cells were generated. From five different mesothelioma cell lines (Thorr 01, Thorr 02, Thorr 03, Thorr 05 and Thorr 06) a preparation was made comprising 50×10⁶ cells/ml. These cells were lysed and the final protein content of the lysate prepared was 15.5 mg protein per ml. The lysate obtained was used for loading the immature dendritic cells (ratio dendritic cells to tumor cell equivalents 3:1). Subsequently, the dendritic cells were matured, frozen and stored.

Administration Protocol

In subsequent cohorts of three patients in total 10×10⁶, 25×10⁶, or 50×10⁶ loaded dendritic cells were administered partly intravenously (IV) and partly intradermally, three times with two weeks interval and after 3 and 6 months to the patients. Primary endpoint was safety. Secondary endpoints were immune responses (delayed type hypersensitivity (DTH) skin testing, peripheral blood testing), response rate (RR), radiographical responses, progression free survival (PFS) and overall survival (OS).

Results

Nine patients (median age 69 years, 8 male, 1 female) were included in the Phase I clinical trial. All patients developed transient grade 1-fever and a grade 1-2 injection site reaction, according to a standard scoring protocol known in the art for monitoring adverse effects of administered pharmaceutical compositions in a clinical trial. No dose limiting toxicities or autoimmunity signs were observed. In 2 patients (22%), both treated with 25×10⁶ loaded dendritic cells, a partial response (PR) was observed, 7 patients had stable disease (SD) as best overall response. All patients were alive and in good clinical condition with a median follow up of 14.6 months after trial inclusion (range 10.3 to 19.2 months). Median OS after diagnosis is 21.3 months. Clearly, the treatment with loaded dendritic cells was safe and had a clinically relevant effect.

Antigen Composition

The antigen composition of the five different types of mesothelioma cell lines used for preparing the lysate was analyzed. This analysis surprisingly showed that the different cells comprised overlapping antigens. In other words, in the different type of mesothelioma cell lines the same antigens are present. From this the present inventors concluded that a strong immune response can be obtained against these cells by using only a limited number of antigens, however, further antigens may be used to possibly further increase the immune response. In view of the abundance of the antigens in the different cell lines the following antigens are preferably used: RAGE1/MOK, Mesothelin, EphA2, Survivin, WT1, MUC1. However, it may be advantageous to also use other antigens in the antigen composition, i.e. RAB38/NY-MEL-1, BING4, MAGE A12, HER-2/Neu, Glypican, LMP2.

TABLE 1
Most relevant antigens present in the model cell lines

| | | | Amount | Amount | Amount | Amount |
|---|---|---|---|---|---|---|
| | | Amount of | of | of | of | of |
| | | antigen | antigen | antigen | antigen | antigen |
| | | expressed in | expressed | expressed | expressed | expresse |
| Antigen | Gene ID | thorr1* | in thorr2* | in thorr3* | in thorr5* | in thorr6 |
| RAGE-1/MOK | 5891 | 1.91 | 10.7 | 50.73 | 310.14 | 48.91 |
| Mesothelin | 10232 | 42.89 | 50.9 | 69.36 | 143.6 | 114.49 |
| EphA2 | 1969 | 32.65 | 97.77 | 24.82 | 62.6 | 162.78 |
| Survivin | 332 | 46.83 | 39.53 | 49.07 | 38.28 | 19.86 |
| WT1 | 7490 | 6.47 | 29.49 | 0.45 | 0.28 | 14.71 |
| MUC1 | 4582 | 10.31 | 12.91 | 11.11 | 18.72 | 12.58 |
| RAB38/NY-MEL-1 | 23682 | 3.21 | 0.27 | 0.48 | 0 | 0.07 |
| BING-4 | 9277 | 20.22 | 17.65 | 37.07 | 24.34 | 34.28 |
| MAGE-A12 | 4111 | 0 | 0 | 51.8 | 0 | 0 |
| HER-2/neu | 2064 | 18.69 | 11.54 | 14.73 | 16.14 | 23.36 |
| glypican-1 | 2817 | 128.93 | 29.62 | 43.47 | 92.31 | 66.31 |
| LMP2 | 5698 | 29.77 | 148.59 | 4.14 | 111.89 | 158.2 |

*FPKM values (fragments per kilobase of exons per million fragments mapped).

Conclusion

Based on the phase I clinical data, it is fair to conclude that dendritic cell immunotherapy with the antigen composition according to the present invention is safe and clinically active. PFS (progression free survival) and OS (overall survival) are improved significantly after treatment. The dose that provided best results was 25×10⁶ loaded dendritic cells, based on the responses in the treated patients and based on logistic reasons (the number of monocytes obtained from the mesothelioma patient during leukapheresis, required for the production of five dosages of loaded autologous dendritic cells). Although a clinically relevant response was observed, it was also observed that the effect was transient and that in at least two patients growth of the tumor started again after several months.

### Example 2: In Vitro Testing of PD-L1 Upregulation of Mesothelioma Cells in the Presence of Interferon Gamma

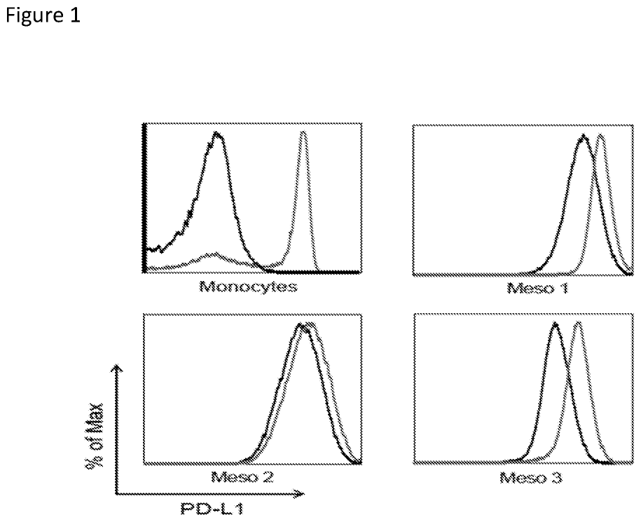

Three malignant mesothelioma cell lines were cultured under identical conditions and their PD-L1 expression was measured using flow cytometry under normal culture conditions (black histograms) and after exposure to IFN-γ (500 IU/ml) overnight (red histograms). As a control, normal human monocytes were also exposed to IFN-γ overnight. The tumour cells that gave rise to the cell lines were originally derived from pleural effusions of mesothelioma patients and the cell lines were established in our laboratory. From FIG. 1 it is clear that malignant mesothelioma cell lines constitutively express PD-L1 which can be marginally upregulated after exposure to IFN-γ (FIG. 1).

### Example 3: Subsequent Treatment of Patients with a PD-1 Inhibitor

Two patients with mesothelioma pre-treated with chemotherapy were progressing after dendritic cell immunotherapy. These patients were included in trial as has been described in example 1.

Upon progression after dendritic cell immunotherapy a PD-L1 staining of a biopsy of both patients was carried out to determine the optimal treatment regime. In both these patients the biopsies showed a high expression of PD-L1. In these patients the diagnostic biopsy (i.e. prior to dendritic cell immunotherapy) proved to be negative for PD-L1.

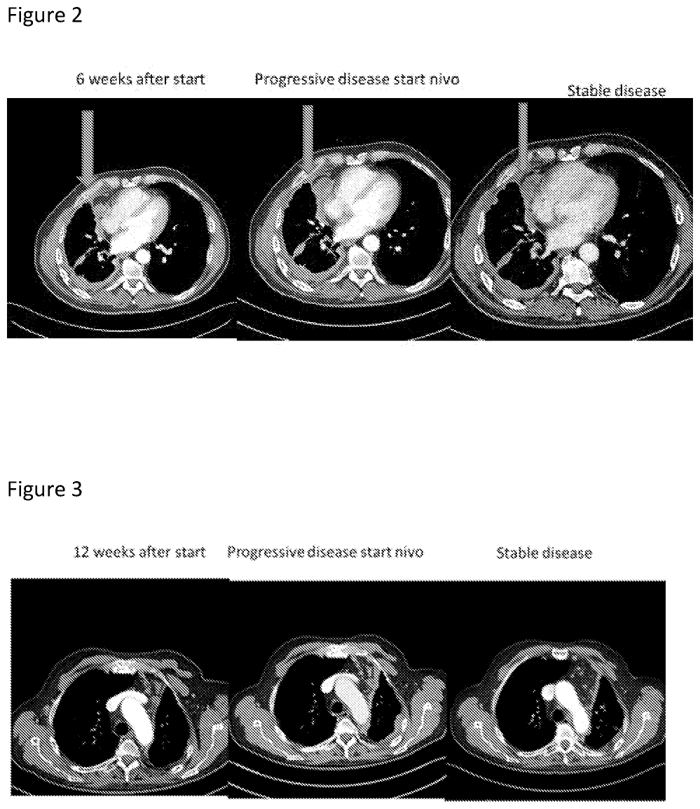

Patient 1: A 60 yrs old male was diagnosed with a malignant mesothelioma epithelial type. He was treated with a total of 4 cycles of platinum pemetrexed with radiographically stable disease as best response. Ten months after the dendritic cell immunotherapy he developed a progressive disease as determined by modified RECIST, after which he was treated with nivolumab 3 mg/kg every 2 weeks. After 6 weeks a new CT scan was made showing a stable disease. The CT scan after 12 weeks showed partial response which was confirmed 6 weeks later. The CT scan images of this patient are depicted in FIG. 2.

Patient 2: A 71 yrs old male was diagnosed with a malignant mesothelioma epithelial type. He was treated with 4 cycles of platinum pemetrexed with a stable disease as best response. Five months after the dendritic cell immunotherapy he developed a progressive disease as determined by modified RECIST. He was treated with nivolumab 3 mg/kg every 2 weeks. After 6 weeks a new CT scan was made showing a partial response which was confirmed 6 weeks later. The CT scan images of this patient are depicted in FIG. 3.

The data above shows that PD-1 treatment after dendritic cell immunotherapy is able to increase immune activity towards a tumor. Hence, it is advantageous to use dendritic cell immunotherapy in combination with checkpoint inhibitors.

1. An antigen composition comprising at least one mesothelioma cancer cell associated antigen and a pharmaceutically acceptable carrier for use in the treatment of cancer, wherein dendritic cells are loaded with said antigen composition and are administered to patients in need thereof together with one or more checkpoint inhibitors.

2. Antigen composition for use according to claim 1, wherein the one or more checkpoint inhibitors inhibit a checkpoint protein selected from the group of: TIM3, CTLA4, PD1, PDL1, PDL2, LAG3, CD137, CD40, OX40, VISTA, CD112R and BTLA, preferably TIM3, CTLA4, PD1, PDL1, PDL2 and LAG3, more preferably PD1 and PDL1

3. Antigen composition for use according to any of the previous claims, wherein the checkpoint inhibitor is chosen from: atezolizumab, avelumab, durvalumab, nivolumab and pembrolizumab, preferably nivolumab.

4. Antigen composition for use according to any of the previous claims, wherein the one or more checkpoint inhibitors are administered simultaneously or sequentially with the antigen loaded dendritic cells, preferably the one or more checkpoint inhibitors are administered after the administration of the antigen loaded dendritic cells.

5. Antigen composition for use according to any of the previous claims, wherein the composition comprises at least two mesothelioma cancer cell associated antigens.

6. Antigen composition for use according to any of the previous claims, wherein the composition comprises at least three, preferably at least five, more preferably at least ten, mesothelioma cancer cell associated antigens.

7. Antigen composition for use according to any of the previous claims, wherein the mesothelioma cancer cell associated antigens are chosen from the group of: RAGE1/MOK, Mesothelin, EphA2, Survivin, WT1, MUC1.

8. Antigen composition for use according to any of the previous claims, wherein the mesothelioma cancer cell associated antigens are chosen from the group of: RAGE1/MOK, Mesothelin, EphA2, Survivin, WT1, MUC1, RAB38/NY-MEL-1, BING4, MAGE A12, HER-2/Neu, Glypican, LMP2.

9. Antigen composition for use according to any of the previous claims, wherein the mesothelioma cancer cell associated antigens are obtained from a lysate of allogenic mesothelioma tumor cells from at least two different cell lines, preferably at least three cell lines, more preferably at least four cell lines, most preferably at least five cell lines.

10. Antigen composition for use according to any of the previous claims, wherein the mesothelioma tumor cell lines are chosen from Thorr 01 (deposit No. DSM ACC3191), Thorr 02 (deposit No. DSM ACC3192), Thorr 03 (deposit No. DSM ACC3193), Thorr 04 (deposit No. DSM ACC3194), Thorr 05 (deposit No. DSM ACC3195), Thorr 06 (deposit No. DSM ACC3196).

11. Antigen composition for use according to any of the previous claims, wherein in patient is administered 1*10⁶ to 1*10⁸ dendritic cells, preferably 1*10⁶ to 50*10⁶ dendritic cells, most preferably 10*10⁶ to 50*10⁶ dendritic cells per vaccination.

12. Antigen composition for use according to any of the previous claims, wherein the dendritic cells are autologous or allogenic, preferably the dendritic cells are autologous.

13. Antigen composition for use according to any of the previous claims, wherein the patients received surgery and/or chemotherapy prior to administration of the loaded dendritic cells and one or more checkpoint inhibitors.

14. Antigen composition comprising at least two mesothelioma cancer cell associated antigens and a pharmaceutically acceptable carrier for use in the treatment of cancer, wherein dendritic cells are loaded with said antigens and are administered to a patient in need thereof, wherein said treatment effectively extends the progression free survival and/or overall survival of the patient.

15. Antigen composition for use according to claim 14, wherein the composition comprises at least three, preferably at least five, more preferably at least ten, mesothelioma cancer cell associated antigens.

16. Antigen composition for use according to any of the claim 14 or 15, wherein the mesothelioma cancer cell associated antigens are chosen from the group of: RAGE1/MOK, Mesothelin, EphA2, Survivin, WT1, MUC1.

17. Antigen composition for use according to any of the claims 14-16, wherein the mesothelioma cancer cell associated antigens are chosen from the group of: RAGE1/MOK, Mesothelin, EphA2, Survivin, WT1, MUC1, RAB38/NY-MEL-1, BINGO, MAGE A12, HER-2/Neu, Glypican, LMP2.

18. Antigen composition for use according to any of the claims 14-17, wherein the mesothelioma cancer cell associated antigens are obtained from a lysate of allogenic mesothelioma tumor cells from at least two different cell lines, preferably at least three different cell lines, more preferably at least four different cell lines, most preferably at least five different cell lines.

19. Antigen composition for use according to claim 18, wherein the mesothelioma tumor cell lines are chosen from Thorr 01 (deposit No. DSM ACC3191), Thorr 02 (deposit No. DSM ACC3192), Thorr 03 (deposit No. DSM ACC3193), Thorr 04 (deposit No. DSM ACC3194), Thorr 05 (deposit No. DSM ACC3195), Thorr 06 (deposit No. DSM ACC3196).

20. Method for treating patients diagnosed with cancer, in particular mesothelioma, by administering to said patients autologous or allogenic dendritic cells loaded with the antigens of the antigen composition according to any of the claim 1-13 or 14-19 and wherein the treatment effectively extends the progression free survival and/or overall survival of the patient.

21. Method according to claim 20, wherein the progression free survival of the patients is extended to at least 14.6 months.

22. Method according to claim 20, wherein the overall survival of the patients is extended with at least 6.3 months when compared to a patient treated with chemotherapy alone.

23. An antigen composition comprising at least two mesothelioma cancer cell associated antigens as referred to in claim 1-13 or 14-19 and a pharmaceutically acceptable carrier.
